(19) United States
(12) Patent Application Publication
(10) Pub. No.: US 2009/0048208 A1
(43) Pub. Date: Feb. 19, 2009

(54) METHOD FOR PRODUCING THE LOW MOLECULAR WEIGHT BETA-GLUCAN BY IRRADIATION AND LOW MOLECULAR WEIGHT BETA-GLUCAN PRODUCED BY THE METHOD

(75) Inventors: Myung-Woo Byun, Jeongeup-si (KR); Ju-Woon Lee, Jeongeup-si (KR); Jong-Il Choi, Yeongduengpo-gu (KR); Jae-Hun Kim, Jeongeup-si (KR); BeomSeok Song, Jungang-gu (KR); EuiHong Byun, Jeongeup-si (KR); NakYun Sung, Yeongi-gun (KR)

Correspondence Address: MERCHANT & GOULD PC, P.O. BOX 2903, MINNEAPOLIS, MN, 55402-0903, US

(73) Assignee: Korea Atomic Energy Research Institute, Yuseong-gu (KR)

(21) Appl. No.: 12/175,515

(22) Filed: Jul. 18, 2008

(30) Foreign Application Priority Data
Jul. 20, 2007 (KR) 10-2007-0072859

(51) Int. Cl. A61K 31/716; C07H 1/00

(52) U.S. Cl. 514/54; 536/123.12

(57) ABSTRACT

Disclosed herein is a method for the preparation of low-molecular weight beta-glucan by irradiation. The low-molecular weight beta-glucan prepared by irradiation shows a random distribution of all beta-glucan structures, low viscosity and high water solubility, and acts as an excellent antioxidant and to activate immune cells, finding useful application in many fields including the food, medical and cosmetics industries.

### BACKGROUND OF THE INVENTION

1. Field of the Invention

The present invention relates to a method for preparing low-molecular weight beta-glucan by irradiation and a low-molecular weight beta-glucan prepared thereby.

2. Description of the Related Art

Irradiation is the process by which an item is exposed to radiation. Irradiation can be intentional, sometimes to serve a specific purpose, for example with the aim of promoting the occurrence of a chemical reaction, disrupting cellular function, or performing sterilization. In the case of food and medical instruments, for example, irradiation is conducted in order to destroy microorganisms, bacteria, viruses, parasites or insects that might be present in the food or on the instruments. Gamma radiation is effective and widely used for these purposes. Gamma radiation is radiation with a single wavelength which can be obtained through the use of radioisotopes, generally Cobalt-60. Showing penetrability strong enough to go through tens of centimeters of concrete, gamma rays are effective for the sterilization of medical instruments even if they are completely packaged. Irradiation using Cobalt-60 is the preferred method for sterilizing food or medical instruments, because the deeper penetration of gamma rays compared to charged particles such as electrons enables administering treatment to liquids or solids in bulk. Korean Patent No. 10-0458965-0000 discloses the manufacture of sterile bovine and porcine plasma protein powders by irradiation with gamma rays at a low dose. In Korean Patent No. 10-0156439-0000, a method is provided for changing physicochemical properties of starch in a stepwise manner by gamma ray irradiation.

Generally, an irradiator apparatus comprises a radiation source assembly in which each of a source capsule containing a radionuclide such as Iridium-192 therein and a connector is provided with internal round threads on its pigtail fitting hole; a source container for storing the radiation source assembly in a shielded state; a control cable assembly, connected with the radiation source assembly, for moving the radiation source assembly; and a source guide tube, connected with the source container, for guiding the radiation source assembly to an inspection subject. Inside the source container are provided a radiation-shielding substance and an S-configured radiation source assembly storage section. Threads are formed at insides of both the control cable assembly and the tube of the source guide tube apparatus, allowing the pigtail to move along therewith in a rotation manner. The control assembly comprises a tube, a gear, a handle, and other components. The structures of the radiation source assembly, the source container, the control cable assembly, and the source guide tube may be typical enough to need no detailed descriptions thereof. Reference may be made to Korean Patent Laid-Open Publication No. 2003-0029317 for detailed description of the structure and operation of such an irradiator apparatus.

Beta-glucans, a kind of polysaccharide, improve the body's immune system's defense capabilities against foreign invaders by enhancing the ability of immune cells to respond to and fight a wide range of challenges, and stimulate the production of interleukin and interferon, both of which activate immune cells. Also, beta-glucans are found to have anti-cancer activity as they stimulate the production of cytokines to enhance the activity of immune cells such as T-cells and B-cells.

There are various kinds of beta-glucans naturally occurring in microbes, mushrooms, and cereal grains. Microbes producing beta-glucans are disclosed in U.S. Pat. No. 5,504,079 for Saccharomyces cerevisiae R4 and in U.S. Pat. No. 5,509,191 for an Agrobacterium sp.

A glucan molecule is a polysaccharide of D-glucose monomers linked by glycosidic bonds, which determine α- and β-type of glucans. Beta 1,3-D glucans are chains of D-glucose molecules, with the six-sided D-glucose rings connected at the 1 and 3 positions. Side chains branch off from the beta-1,3 glucan backbone at position 4 (1,4 linked) or position 6 (1,6 linked). Some researchers have suggested that it is the frequency, location, and length of the side-chains rather than the backbone of beta-glucans that determine their immune system activity. Beta-glucans naturally occur and are generally distributed over a wide range of organisms including mushrooms and yeasts, and range in molecular weight from kilo-daltons to mega-daltons (H Yamada, Paulsen, B. S. (Ed), Proceedings of the Phythochemical Society of Europe, vol. 44, p. 15, 2000). Beta-glucans are used on a small scale in the food industry because of the gelling ability and high viscosity thereof (Dawkins, N. L. et. al., Food hydrocolloids 9, 1-7 1995). Beta-glucans have been given a GRAS (Generally Recognized As Safe) rating by the FDA in 1983 (sanctioned in Title 21, vol 3).

When administered orally, beta-glucans show significantly poor absorbability. This is because beta-glucans are macro-macromolecules formed through crosslinks of the backbones. Beta-glucans are insoluble in water and show antioxidant activity. The large molecular weights of beta-glucans thereof act as an obstacle to the absorption thereof into the stomach. The poor absorbability of large-molecular weight beta-glucans is attributed to the lack of a certain enzyme in a digestive pathway of the stomach. Thus, it is difficult for water-insoluble, large-molecular weight glucans to bind to certain receptors and be absorbed into cells at a concentration large enough for effective immunomodulation.

In addition, the high viscosity thereof makes it difficult to use beta-glucans as functional food additives. For example, beta-glucans with high viscosity and low solubility poses several impediments including retardation of malting, poor wort separation, difficulties in beer filtration, salad dressing and icecream formulation, formation of undesirable precipitations (Carr, J. M. et. al., Cereal Chem. 67, 226, 1990).

The difficulty in using beta-glucans as food additives is attributed to the high viscosity thereof (Wood, P. J., Webster, F. H. (Ed.), Oats: Chemistry and technology, pp. 121-152, 1986). The high viscosity of beta-glucans is relevant to the high molecular weights and concentrations thereof (Beer, M. U. et. al., Cereal chemistry 73, 58-62. 1996). Thus, a limitation on molecular weights is needed.

For use in foods and medicines, beta-glucans must have not only high water solubility and absorbability, but also effective immune activity. Thus, a method for preparing beta-glucans which are relatively small in molecular weight and soluble in water, which optimally bind to suitable receptors, and are absorbable by the inner wall of the stomach is needed. Also, low-molecular weight beta-glucans, serving as immunomodulators with high immune activity, are required.

For reducing the molecular weights of beta-glucans, previous studies suggested acidic hydrolysis (Hasegawa et al., Carbohydr. Polym. 20(4), 279-283, 1993), enzymatic treatment, and physical treatment (Ilyina et al., Process Biochem. 35(6), 563-568, 2000). However, these methods suffer from the disadvantage of requiring a filtration process because additives are used in early reactions and side products are formed. In contrast, gamma ray irradiation is simpler and more environmentally friendly than the conventional methods because it does not require the use of additives in early reactions, nor does it form side products.

Gamma irradiation is used for the final biological sterilization (Hugo, Internat. Biodet. Biodegrad. 36(3.4), 197-217, 1995) of materials that can be subsequently used for manufacturing biomedical products. The basic advantages of degradation of polymers by radiation include the ability to promote changes reproducibly and quantitatively, with neither the introduction of chemical reagents, nor the need for special equipments/setup to control for temperature, environment, and additives (Charlesby, Radiat. Phys. Chem. 18 (1.2), 59-66, 1981).

Hence, the aim of the present invention is to apply radiation technology to beta-glucan to reduce its molecular weight and viscosity while increasing the water solubility and biological safety thereof, without producing disadvantages in structural properties thereof.

Leading to the present invention, intensive and thorough research into the low molecularization of beta-glucans, resulted in the finding that the irradiation of gamma rays to beta-glucan solutions causes beta-glucans to undergo degradation in molecular weight so as to form low-molecular weight beta-glucans which are high in water solubility and reducing content and also which exhibit improved antioxidation activity, spelenocyte proliferation, and cytokine stimulation.

### SUMMARY OF THE INVENTION

Accordingly, the present invention has been made keeping in mind the above problems occurring in the prior art, and an object of the present invention is to provide a method for preparing low-molecular weight beta-glucan, which is able to promote the degradation of large-molecular weight beta-glucan reproducibly and quantitatively, without need for introduction of chemical reagents, and special equipments/setup to control for temperature, environment, and additives.

It is another object of the present invention to provide low-molecular weight beta-glucan in which all structures of beta-glucans including beta-1,3-glucans, beta-1,4-glucans and beta-1,6-glucans are randomly distributed.

In order to accomplish the above objects, the present invention provides a method for preparing low-molecular weight beta-glucan by irradiation.

Also, the present invention provides low-molecular weight beta-glucan prepared by the above method of the present invention.

Also, the present invention provides an immune-enhancing, pharmaceutical composition comprising the low-molecular weight beta-glucan.

Also, the present invention provides a method for enhancing immunity, comprising administering the low-molecular weight beta-glucan at a pharmaceutically effective dose to a subject in need thereof.

Also, the present invention provides the use of the low-molecular weight beta-glucan in the preparation of an immune-enhancing, pharmaceutical composition.

Also, the present invention provides an antioxidant pharmaceutical composition comprising the low-molecular weight beta-glucan as an active ingredient.

Also, the present invention provides a method for antioxidation treatment, comprising administering the low-molecular weight beta-glucans at a pharmaceutically effective dose to a subject in need thereof.

Also, the present invention provides the use of the low-molecular weight beta-glucan in preparation of an antioxidant pharmaceutical composition.

Also, the present invention provides an immune-enhancing health food, comprising the low-molecular weight beta-glucan as an active ingredient.

Also, the present invention provides the use of the low-molecular weight beta-glucan in preparation of an immune-system enhancing health food.

Also, the present invention provides a functional cosmetic for preventing and treating atopy, comprising the low-molecular weight beta-glucan as an active ingredient.

Also, the present invention provides a method for preventing and treating atopy, comprising the low-molecular weight beta-glucan at a cosmetically effective amount to a subject in need thereof.

Also, the present invention provides the use of the low-molecular weight beta-glucan in preparation of a functional cosmetic for preventing and treating atopy.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing the effect of gamma ray irradiation on the molecular weight of beta-glucan,

FIG. 2 is a graph showing the effect of gamma ray irradiation on the viscosity of beta-glucan,

FIG. 3 is a graph showing the effect of gamma ray irradiation on the solubility of beta-glucan,

FIG. 4 is a graph showing the effect of gamma ray irradiation on the content of reducing sugars of beta-glucan,

FIG. 5 is a graph showing the effect of gamma ray-irradiated beta-glucan on in vitro T cell activation,

FIG. 6 is a graph showing the effect of gamma ray-irradiated beta-glucan on the in vitro secretion of IFN-γ from splenocytes,

FIG. 7 is a graph showing the effect of gamma ray-irradiated beta-glucan on the in vitro secretion of IL-2 from splenocytes,

FIG. 8 is a graph showing the antioxidant activity of gamma ray-irradiated beta-glucan against 2,2-Diphenyl-1-picryl-hydrazyl (DPPH) radicals,

FIG. 9 is a graph showing the effect of gamma ray-irradiated beta-glucan on in vivo T cell activation,

FIG. 10 is a graph showing the effect of gamma ray-irradiated beta-glucan on the in vivo secretion of IFN-γ from splenocytes, and

FIG. 11 is a graph showing the effect of gamma ray-irradiated beta-glucan on the in vivo secretion of IL-2 from splenocytes.

### DESCRIPTION OF THE PREFERRED EMBODIMENTS

In accordance with an aspect thereof, the present invention pertains to a method for preparing low-molecular weight beta-glucans, comprising:

1) dissolving beta-glucans in a solvent; and

2) exposing the beta-glucan solution of Step (1) to radiation.

The beta-glucans to be used in Step (1) of the method may be purchased or obtained from microbes, mushrooms or cereals.

Examples of the solvent useful in Step (1) include, but are not limited to, distilled water, buffers, culture media and alcohols, with a preference for distilled water.

The radiation used in Step (2) may be gamma rays, electron rays or X-rays, with a preference for gamma rays (Dauphin J F et. al., Elsevier Scientific, pp. 131-220).

Gamma ray sources may be Co-60, Kr-85, Sr-90 or Cs-137, but are not limited thereto, with a preference for Co-60. In accordance with the present invention, for example, gamma rays may be preferably irradiated onto the solution at a radiation absorbed dose of 10-100 kGy, and preferably at a radiation absorbed dose of 30-50 kGy. When exposed to a radiation absorbed dose greater than 100 kGy, beta-glucans are degraded into excessively low molecular weights which are so small that desired immune activity is not achieved. At a dose smaller than 10 kGy, the degradation of beta-glucans is slight.

In accordance with the present invention, the beta-glucans prepared using the method range in molecular weight from 1 kDa to 100 kDa. Beta-glucans with a molecular weight of 170 kDa or larger were found to be degraded to molecules with a molecular weight of 30-60 kDa when irradiated with gamma rays at a dose of 10 or 30 kGy and with a molecular weight of 25 kDa or less when irradiated with gamma rays at a dose of 50 kGy or larger. Thus, when exposed to a gamma ray, the polysaccharides beta-glucans can be partially degraded and thus undergo low molecularization.

In accordance with another aspect thereof, the present invention pertains to the low-molecular weight beta-glucans prepared using the method.

Useful as it is in the low molecularization of beta-glucan by degrading beta-1,3-glucans and beta-1,4-glucans, treatment with enzymes finds great difficulty in cleaving beta-1,6-glucans. In contrast, irradiation randomly degrades all beta-glucan structures. Thus, the beta-glucans degraded by irradiation have molecular structures different from those obtained through enzyme treatment (Ilyina, A. V. et. al., Process Biochem. 35(6), 563-568, 2000; Shimokawa, T. et. al., Biosci. Biotech. Biochem. 60(9), 1532-1534, 1996).

In experimental examples of the present invention, the beta-glucans prepared according to the method of the present invention were measured for viscosity, water solubility and the presence of reducing sugars. The viscosity of the beta-glucans was decreased with an increase in the radiation dose and became significantly low at a dose of 50 kGy. It is known that the viscosity change of the polysaccharides is attributed to cleavage of polysaccharide molecules, which is dependent on the dose of gamma radiation used. Also, the irradiated beta-glucans increased in water solubility with an increase in the radiation dose and the solubility became significantly high at a radiation absorbed dose of 50 kGy. The solubility increase is attributed to the fact that irradiation by gamma rays destroys and degrades the molecular structure of beta-glucans to form low-molecular weight particles (Graham, J. A. et. al., Journal of Science Food Agriculture 82, 1599-1605, 2002). In addition, the presence of reducing sugars of the exposed beta-glucans increased with an increase in the radiation dose and become significantly high at a radiation absorbed dose of 50 kGy. An increase in the level of reducing sugars means an increase in the number of the terminal regions, that is, of the reducing regions of sugar, resulting from the radiation-induced breakdown of polymers. Accordingly, the increase in the content of reducing sugar is attributed to the formation of low-molecular weight saccharides, such as glucose, as a result of depolymerization.

The low-molecular weight beta-glucans prepared according to the method are characterized by the fact that all structures of beta-glucans including beta-1,3-glucans, beta-1,4-glucans and beta-1,6-glucans are randomly fragmented. Enzyme treatment easily degrades beta-1,3-glucan or beta-1,4-glucan structures, but cannot bring about a cleavage of beta-1,6-glucan structures. However, irradiation can easily degrade beta-1,6-glucan structures as well.

In experimental examples of the present invention, when exposed to gamma radiation, beta-glucans were measured and were found to have a decreased content in all of beta-1,3-glucan, beta-1,4-glucan and beta-1,6-glucan. However, it is difficult to effectively hydrolyze beta-1,6-beta-1,3-glucans with acids or enzymes (Shin, H. J. et. al., J. biotechnol. Bioeng. 18(5), 352-355, 2003). However, randomly degraded beta-glucans can be obtained by irradiation.

In accordance with a further aspect thereof, the present invention pertains to a pharmaceutical composition for immune enhancement, comprising the as an active ingredient low-molecular weight beta-glucans prepared using the method.

In accordance with still a further aspect thereof, the present invention pertains to a method of enhancing immunity, comprising administering the low-molecular weight glucans at a pharmaceutically effective dose.

In accordance with still another aspect thereof, the present invention pertains to the use of the low-molecular weight beta-glucans in the preparation of a pharmaceutical composition for immune enhancement.

The low-molecular weight beta-glucans prepared according to the preparation method of the present invention have the ability to activate spelenic T cells. In vivo and in vitro assays demonstrated that the T-cell activation of irradiated beta-glucans increased with an increase in radiation dose. Particularly, the beta-glucans exposed to a radiation absorbed dose of 50 kGy were found to have significantly increased T cell activity.

Also, the low-molecular weight beta-glucans prepared according to the preparation method of the present invention stimulate the secretion of cytokines from splenocytes. The irradiated beta-glucans were found to significantly increase the release of cytokines following an increase in radiation dose as revealed by in vivo and in vitro measurements for the level of IFN-γ and IL-2, the cytokines released from Th 1.

Therefore, the low-molecular weight beta-glucans prepared according to the present invention exhibit high immunomodulation functions.

In accordance with yet a further aspect thereof, the present invention pertains to an antioxidant composition comprising the low-molecular weight beta-glucans as an active ingredient.

In accordance with yet another aspect thereof, the present invention pertains to a method for antioxidation treatment, comprising administering the low-molecular weight beta-glucans at a pharmaceutically effective dose to a subject in need thereof.

In accordance with yet still a further aspect thereof, the present invention pertains to the use of the low-molecular weight beta-glucans in the preparation of antioxidant pharmaceutical composition.

Particularly, the low-molecular weight beta-glucans prepared according to the preparation method of the present invention have a high DPPH radical-scavenging function. The low-molecular weight beta-glucans of the present invention were found to significantly increase in antioxidant activity following an increase in radiation dose as revealed by scavenging DPPH radicals.

For application to medicines, the low molecular weight beta-glucan of the present invention may be used in combination with one or more active ingredients identical or similar in function therewith.

The low-molecular weight beta-glucan may be in the form of a common drug agent which is administrable via oral or non-oral routes. That is, the low-molecular weight beta-glucan may be administered in various oral or non-oral dosage forms for clinical practice. In this regard, the low-molecular weight beta-glucan of the present invention may be usually formulated in combination with a diluent or excipient, such as a filler, a thickening agent, a binder, a wetting agent, a disintegrant, a surfactant, etc.

Solid preparations intended for oral administration of the compound of the present invention may take the form of tablets, pills, powders, granules, capsules, and the like. In regards to these solid agents, the low-molecular weight beta-glucan of the present invention is formulated in combination with at least one excipient such as starch, calcium carbonate, sucrose, lactose, or gelatin. In addition, a lubricant such as magnesium stearate, talc, or the like may also be added. Liquid preparations intended for oral administration include suspensions, internal use solutions, emulsions, syrups, and the like. In addition to simple diluents such as water or liquid paraffin, various excipients, such as wetting agents, sweetening agents, aromatics, preservatives, and the like may be contained in the liquid preparations. Also, the low-molecular weight beta-glucan of the present invention may be administered via a non-oral route. For this, sterile aqueous solutions, non-aqueous solvents, suspensions, emulsions, lyophilizates, suppositories, and the like may be used. Injectable propylene glycol, polyethylene glycol, vegetable oils such as olive oil, and esters such as ethyl oleate may be suitable for non-aqueous solvents and suspensions. The basic materials of suppositories include Witepsol, macrogol, Tween 61, cacao butter, laurin butter, glycerol, and glycerogelatin.

The effective dosage of the low-molecular weight beta-glucan in accordance with the present invention depends on various factors, including the patient's weight, age, gender, state of health and severity of disease, diet, time of administration of the drug, route of administration, excretion rate, etc. The low-molecular weight beta-glucan according to the present invention may be administered in a single dosage or may be divided into two to six dosages per day at a daily dosage ranging from 0.1 to 100 mg/kg, preferably from 30 to 86 mg/kg and more preferably from 50 to 60 mg/kg.

The low-molecular weight beta-glucan of the present invention may be used alone or in conjunction with other agents for surgical operation, radiotherapy, hormonal therapy, chemical therapy or biological modulation.

In accordance with yet still another aspect thereof, the present invention provides a health food composition for enhancing immunity, comprising the low-molecular weight beta-glucan as an active ingredient.

In accordance with additional a further aspect thereof, the present invention provides the use of the low-molecular weight beta-glucan in the preparation of a health food composition for enhancing immunity.

As a food additive, the low-molecular weight beta-glucan of the present invention may be properly used alone or in combination with other food ingredients according to conventional methods. The amount of the active ingredient used in accordance with the present invention may vary depending on the purpose thereof (prevention, health improvement, therapeutic treatment). Generally, when used for the preparation of foods or beverages, the active ingredient according to the present invention may be added in an amount of 15 weight % or less based on the total weight of the health food and preferably in an amount of 10 weight % or less. In the case where the active ingredient is applied to health foods which are designed to be taken habitually, its content may be below the above-mentioned range. However, the low-molecular weight beta-glucan does not have the problem of being harmful to the body and thus can be used in an amount exceeding the ranges specified.

No particular limitations are imposed on the kind of foods to which the low-molecular weight beta-glucan can be added. Examples of such foods include meats, sausages, breads, chocolates, candies, confectioneries, pizzas, ramen noodles and other noodles, gum, dairy products such as ice cream, various soups, beverages, teas, drinks, alcoholic beverages, vitamin complexes, and other healthy food supplements and are not limited to the ones mentioned. All typically accepted health foods may contain the active ingredient according to the present invention.

A healthy beverage composition according to the present invention may further contain various fragrant or natural carbohydrates. Examples of such natural carbohydrates include monosaccharides, such as glucose and fructose, disaccharides such as maltose and sucrose, polysaccharides such as dextrin and cyclodextrin, and sugar alcohols such as xylitol, sorbitol and erythritol. Also, sweeteners, e.g., natural sweeteners such as thaumatin and a stevia extract, or synthetic sweeteners such as saccharin and aspartame, may be added to the health food to which the active ingredient of the present invention is applied. The natural carbohydrate may be used in an amount of approximately 0.01˜0.04 grams based on 100 mL of the beverage composition of the present invention, and preferably in an amount of approximately 0.02˜0.03 grams.

In addition, the health food composition of the present invention may contain various nutrients, vitamins, minerals, electrolytes, flavors, colorants, pectic acid and salts thereof, alginic acid and salts thereof, organic acids, protective colloidal thickeners, pH modifiers, stabilizers, antiseptics, glycerin, alcohols, and carbonating agents used in carbonated beverages. Moreover, the composition of the present invention can contain fruit flesh for preparing natural fruit juices, fruit beverages and vegetable beverages. These ingredients may be used individually or in combination. The ratio of these additives is not important, but is generally selected in a range of 0.01 to 0.1 weight parts per 100 weight parts of the composition of the present invention.

In accordance with additional another aspect thereof, the present invention pertains to a functional cosmetic for preventing and treating atopy which comprises the low-molecular weight beta-glucan of the present invention as an active ingredient.

In accordance with additionally yet another aspect thereof, the present invention pertains to a method for preventing and treating atopy, comprising administering the low-molecular weight beta-glucan at a cosmetically effective dose to a subject in need thereof.

In accordance with additionally yet a further aspect thereof, the present invention pertains to the use of the low-molecular weight beta-glucan in the preparation of a functional cosmetic for preventing and treating atopy.

Having immune enhancement activity, beta-glucan shows dermatitis relieving and moisturizing effects which are useful in the prevention and improvement of atopy (pillai, R. et. al., Research Disclosure 499. 1278-1279, 2005). Because it can easily pass through the skin thanks to the low molecular weight thereof, the beta-glucan of the present invention can be used as an active ingredient of a cosmetic for preventing and treating atopy.

A cosmetic comprising the low-molecular weight beta-glucan of the present invention as an active ingredient may be formulated as a general emulsion type or as a water-soluble type form. Examples of the emulsion-type cosmetics include nutrition lotions, creams, essences and the like. A skin lotion is a kind of water-soluble cosmetic.

Examples of the cosmetic forms to which the beta-glucan of the present invention is applicable include solutions, gels, solid or paste preparations, oil-in-water emulsions, suspensions, microemulsions, microgranules or ionic liposomes, non-ionic vesicle dispersions, creams, skins, lotions, powders, ointments, sprays, conceal sticks, etc. Also, it may be prepared into a foam form or an aerosol form having a quantity of compressed propellant.

In addition to the low-molecular weight beta-glucan of the present invention, the cosmetic preparation may additionally comprise lipids, organic solvents, dissolving agents, thickening agents, gelling agents, softeners, anti-oxidants, suspending agents, stabilizers, foaming agents, aromatics, surfactants, water, ionic or non-ionic emulsifiers, fillers, sequestering agents, chelating agents, preservatives, vitamins, UV blocking agents, wetting agents, essential oils, dyes, pigments, hydrophilic or lipophilic activators, liposomes, and/or other general supplements used in the skin science field. These ingredients may be used in amounts that are generally accepted in the skin science field.

As described above, the low-molecular weight beta-glucans prepared by irradiation in accordance with the present invention exhibit such physical properties as reduced viscosity and increased water solubility and such biological properties as increased antioxidant activity, splenocyte stimulation and cytokine secretion, and thus find useful application in many fields including the food, medical and cosmetics industries.

A better understanding of the present invention may be obtained through the following examples which are set forth for purposes of illustration, but are not to be construed as to limit the present invention.

### Example

### Preparation of Low-Molecular Weight Beta-Glucans by Gamma Ray Irradiation

Beta-glucans were purchased as a white powder with a polysaccharide content of 97.2% and a beta-glucan content of 80% or higher from ACE BIOTECH (Korea). The beta-glucan was dissolved in distilled water to give a 10% (w/v) beta-glucan solution.

Gamma irradiation was carried out using a cobalt-60 irradiator in the Advanced Radiation Technology Institute, Korea Atomic Energy Research Institute (Jeoung-Eup, Korea). The strength of the source was approximately 100 kCi with a dose rate of 10 kGy/h. Dosimetry was conducted using 5 mm diameter alanine dosimeters (Bruker Instruments, Rheinstetten, Germany). The dosimeters were calibrated against an international standard set by the International Atomic Energy Agency (IAEA). The dosages applied in this experiment were 0, 10, 30, and 50 kGy at room temperature.

### Experimental Example 1

### Measurement of Molecular Weight

After gamma ray irradiation, the molecular weights of the beta-glucans were measured through gel permeation chromatography (GPC). A Waters GPC system equipped with a Waters 515 pump, a 2× PLaqagel-OH mixed (7.8×300 mm) column, and a Waters 2410 refractive index detector was used for performing the GPC. Each sample solution was diluted with distilled water to obtain a concentration of 10 mg/mL and 100 μL of the diluted solution was loaded onto the GPC system. The column was operated at 40° C. and eluted with distilled water at a flow rate of 1.0 mL/min. The column was calibrated using various standard dextrans at a concentration of 0.1% (w/w). Analysis was completed using the Millennium software, version 3.05.01.

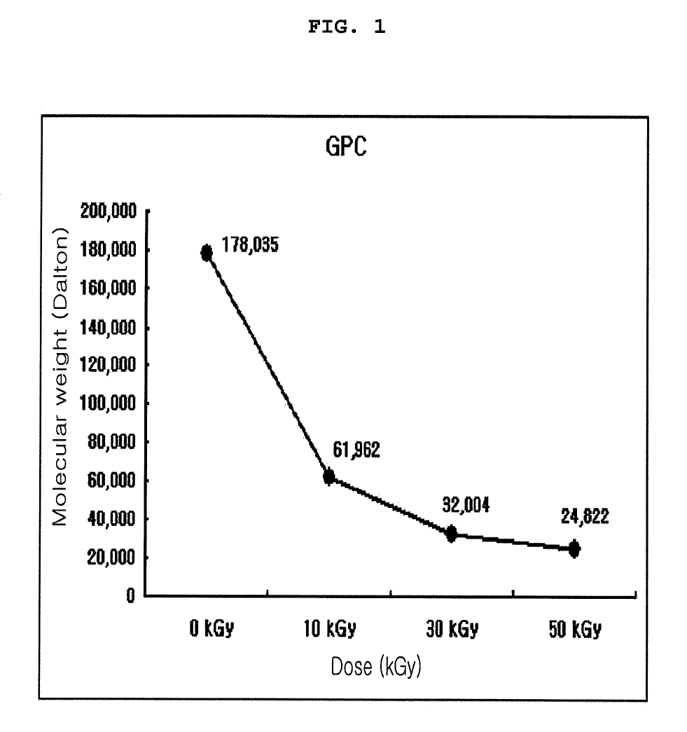

With reference to FIG. 1, the effect of gamma ray irradiation on the molecular weight of beta-glucan is shown. As seen in the graph, the average molecular weight of beta-glucan in solution is 178.035 KDa in the non-irradiated sample, whereas it was decreased to 61,962 KDa, 32,004 KDa, 24,822 KDa upon irradiation at doses of 10, 30, and 50 kGy, respectively. It has been reported that beta-glucans with a molecular weight of 100 kDa are considered as high molecular weight compounds, and the low molecular weight range is considered to be below 50 kDa (Bohn, J. A. et. al., Carbohydrate Polymers. 28(1), 3-14, 1995). This gamma ray-induced change might be due to the breakage of the glycosidic bond of the polysaccharide, as the loss of molecular weight attributed to the glycosidic bond breakage was reported to result in the formation of low-molecular weight sugars (Sokhey, A. S. et. al., Food Struct. 12, 397-410, 1983).

### Experimental Example 2

### Viscosity Measurement

The viscosity of the beta-glucan solution was measured at room temperature using a Brookfield viscometer (DV-II+pro, Brookfield Engineering Laboratories, MA, USA) with the S21 spindle at 180 rpm.

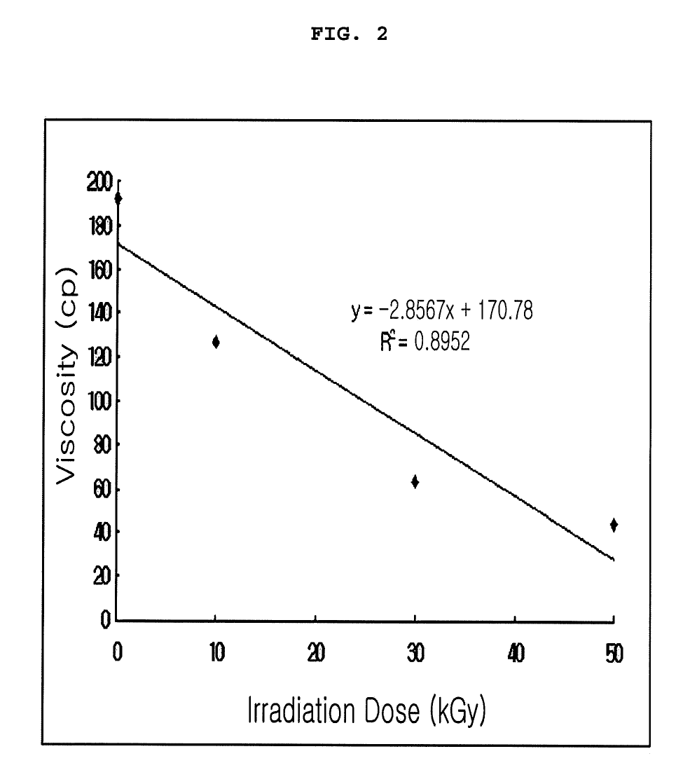

FIG. 2 shows the effect of gamma ray irradiation on the viscosity of beta-glucan. As seen in this graph, the beta-glucan solution was found to increase in viscosity alongside an increase in the absorbed radiation dose. The viscosity of the non-irradiated sample was 191.93 cp and was decreased to 135.5 (cp) and 75.81 (cp), respectively, when irradiated at doses of 10 kGy and 30 kGy and further reduced to 43.9 (cp) by a dose of 50 kGy.

### Experimental Example 3

### Water Solubility Measurement

In order to determine the solubility difference incurred by the irradiation treatment of beta-glucan, the sample solutions were lyophilized and then vortexed with deionized water for 20 min. After centrifugation at 3500 rpm for 20 min, the supernatant thus formed was dried at 100° C. The dried products were weighed. The solubility was calculated as follows:

$$\% Solubility = \frac{Wt . of Dried Supernatant}{Wt . of Lyophilized Sample} \times 100$$

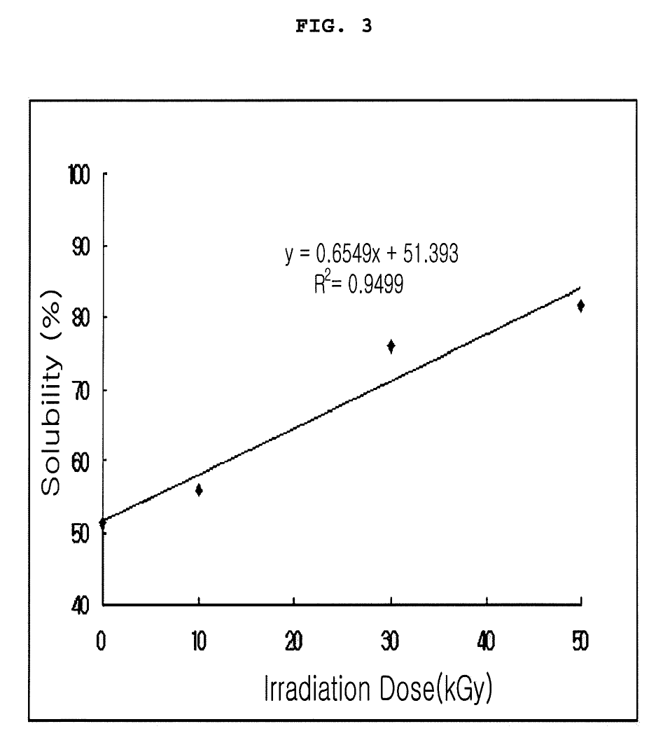

With reference to FIG. 3, the effect of gamma ray irradiation on the solubility of beta-glucan is shown. Non-irradiated beta-glucan was found to be dissolved in water to about 51.38% while the solubilities of beta-glucan irradiated at 10 and 30 kGy were increased to 55.76% and 75.81%, respectively. As compared to the non-irradiated sample, the greatest increment of water solubility was detected in the 50 kGy-irradiated sample which showed a solubility of 81.72%.

### Experimental Example 4

### Reducing Sugar Content

Reducing sugar was measured using a 3,5-dinitrosalicylic acid (DNSA) method (Miller, G. L. 1959). 1 mL of the beta-glucan sample was placed in a 15 ml tube and mixed with 2 ml of the DNSA reagent (a solution of 0.5 g dinitrosalicylic acid, 8 g sodium hydrate and 150 g rochell salt in 500 mL deionized water) for 5 sec. After reaction at 90° C. for 10 min, the reaction mixture was cooled. The samples were measured for reducing sugar level using a spectrophotometer (UV-1601 PC, Shimadzu Co., Tokyo, Japan) at 550 nm.

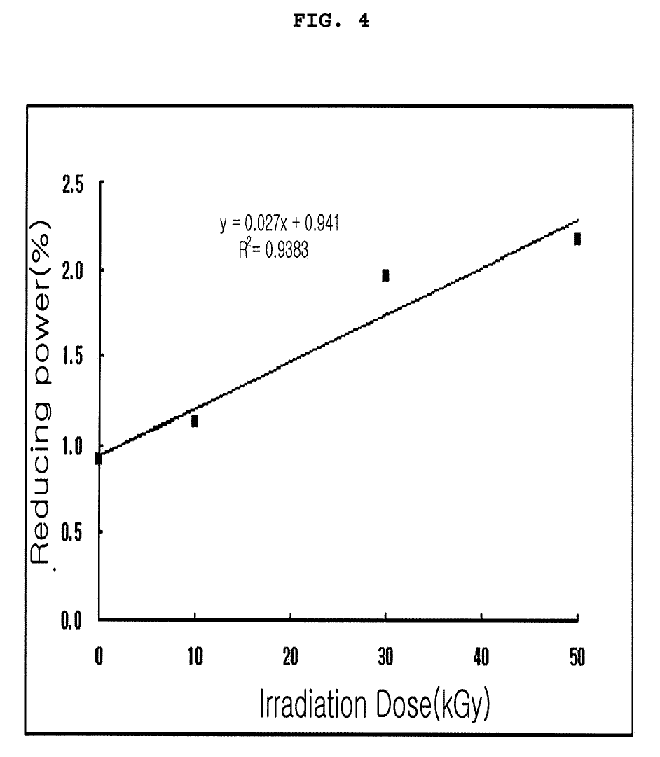

In FIG. 4, reducing sugar contents of gamma ray-irradiated beta-glucan are shown. At R²=0.9383, as seen in FIG. 4, the reducing sugar content was measured to be 0.917% for the non-irradiated beta-glucan and was increased to 1.128% and 1.973% when it was irradiated at doses of 10 and 30 kGy, respectively. As compared to the non-irradiated sample, the greatest portion of reducing sugar content was detected in the 50 kGy-irradiated sample which had a reducing sugar content of 2.173%.

### Experimental Example 5

### Structural Change of Beta-Glucan

Gamma ray-irradiated beta-glucan was dissolved in conc. HCl (37%, 10N) and subjected to acid hydrolysis at 100° C. for 2 hours in 1.3 N HCl. The acid-hydrolyzed beta-glucan was measured for the content of beta-1,3-glucan, beta-1,4-glucan and beta-1,6-glucan using a beta-D-glucose assay kit (purchased from Megazyme International Ireland Ltd.).

In Table 1, contents of beta-1,3-glucan, beta-1,4-glucan and beta-1,6-glucan in the beta-glucan low-molecularized by irradiation are summarized.

TABLE 1
Contents of Beta-1,3-Glucan, Beta-1,4-Glucan and Beta-1,6-Glucan
in Gamma Ray-Irradiated Beta-Glucan

| | Beta-Glucan (%) | | | |
|---|---|---|---|---|
| Absorbed Dose | Beta-1,3- | Beta-1,4- | Beta-1,6- | Total |
| (kGy) | Glucan | Glucan | Glucan | (%) |
| 0 | 35.79 | 36.24 | 11.01 | 83.04 |
| 10 | 30.21 | 30.43 | 9.50 | 70.14 |
| 30 | 25.96 | 25.81 | 8.66 | 60.43 |
| 50 | 24.88 | 24.14 | 7.78 | 56.80 |

As is apparent from the data of Table 1, the low-molecularized beta-glucan obtained by irradiation was decreased in the content of all beta-1,3-glucan, beta-1,4-glucan and beta-1,6-glucan. Therefore, the data indicate that irradiation cleaves the glycidic bonds of beta-glucan in a random mode.

### Experimental Example 6

### In Vitro Assay of Gamma Ray-Irradiated Beta-Glucan for T Cell Activation

An MTT (3-[4,5-dimethylthiazol-2-yl]-2,5-diphenyl tetrazolium bromide) assay was conducted for determining splenocyte proliferation. After incubation for 24 hours, splenocytes were added with 30 μL of a 5 mg/mL MTT solution in PBS and incubated at 37° C. for an additional 2 hours. After centrifugation, the cell pellets were dissolved in 100 μL of dimethylsulfoxide (DMSO, Sigma) and incubated at 37° C. for 5 min. Absorbance at 595 nm was measured using a microplate ELISA reader.

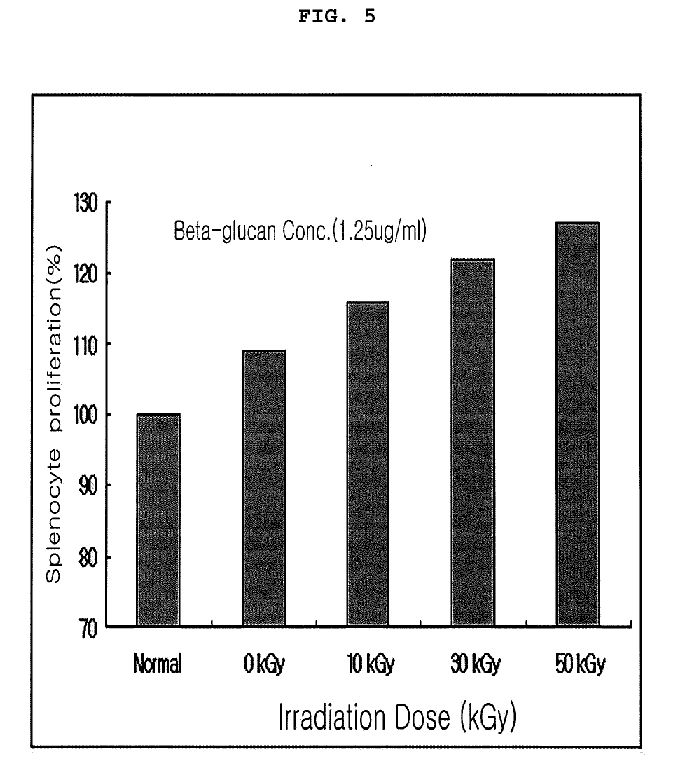

With reference to FIG. 5, the effect of gamma ray-irradiated beta-glucan on T cell activation is shown. The activity of T cells of mice was analyzed through MTT assay with splenic cells treated for 24 hours with gamma ray-irradiated or non-irradiated beta-glucan. The splenocytes were observed to greatly increase in T cell activity when incubated in the presence of gamma ray-irradiated beta-glucan, compared to those incubated in the presence of non-irradiated beta-glucan. Splenic T cell activity was measured to be 109, 116, 122 and 127% when mouse splenocytes were treated with beta-glucan irradiated with gamma rays at doses of 0, 10, 30 and 50 kGy, respectively. The highest splenic T cell activity was detected in the spenocytes treated with 50 kGy irradiated beta-glucan.

### Experimental Example 7

### In Vitro Assay of Gamma Ray Irradiated Beta-Glucan for Cytokine Secretion

For cytokine analysis, single cell suspensions of spleen cells were plated at a density of 1×10⁶ cells/well onto 96-well plates containing an RPMI-1640 medium supplemented with 10% fetal bovine serum (FBS), 100 U/mL penicillin and 100 U/mL streptomycin in each well, and incubated at 37° C. in a 5% CO₂ atmosphere. The cells were treated with 0˜2.5 μg/mL of beta-glucan before incubation for 24 hours. After incubation, the cells were stored at −70° C. until analysis.

In an in vitro system, irradiated and non-irradiated beta-glucans were assayed for immune activity via Th1 cytokines. T cells (cytotoxic and Th1) and natural killer (NK) cells secrete interferon gamma (IFN-γ), which serves mainly as a macrophage-activating factor. Changes in Th1 cytokines were analyzed through an ELISA method using IL-1 and IFN-γ antibodies and a BD OptEIA™ set (BD Biosciences, San Jose, Calif.).

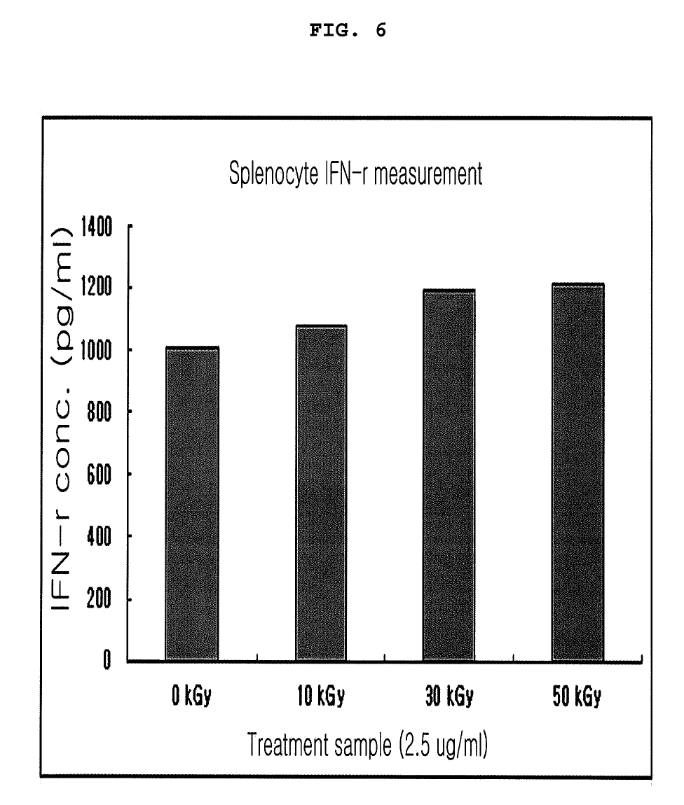

With reference to FIG. 6, the levels of Th1-released IFN-γ are shown according to stimulation with non-irradiated and irradiated beta-glucan. As seen in this figure, the secretion of the Th1 cytokine IFN-γ was increased with an increase in radiation dose. That is, the beta-glucan irradiated at a dose of 30 or 50 kGy was found to stimulate the release of a greater amount of the cytokine than did the beta-glucan irradiated at a dose of 0 or 10 kGy.

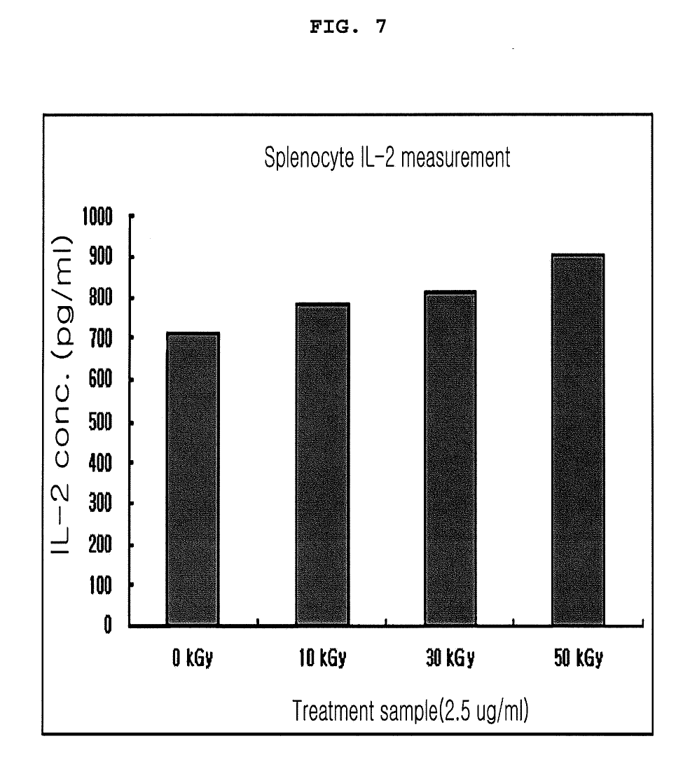

FIG. 7 shows the levels of Th1-released IL-2 in splenocytes according to stimulation with non-irradiated and irradiated beta-glucan. Like IFN-γ, the Th1 cytokine IL-2 was increased in secretion level alongside an increase in the radiation dose. That is, the beta-glucan irradiated at a dose of 30 or 50 kGy was found to stimulate the release of a greater amount of the cytokine than did the beta-glucan irradiated at a dose of 0 or 10 kGy.

### Experimental Example 8

### In Vitro Assay of Gamma Ray-Irradiated Beta-Glucan for DPPH (2,2-Diphenyl-1-picryl-hydrazyl) Radical Scavenging Ability

The ability of gamma ray-irradiated beta-glucan to scavenge radicals was analyzed according to the Amarowicz method (Amarowicz, R. et. al., Food Chemistry 84, 4, 551-562, 2004).

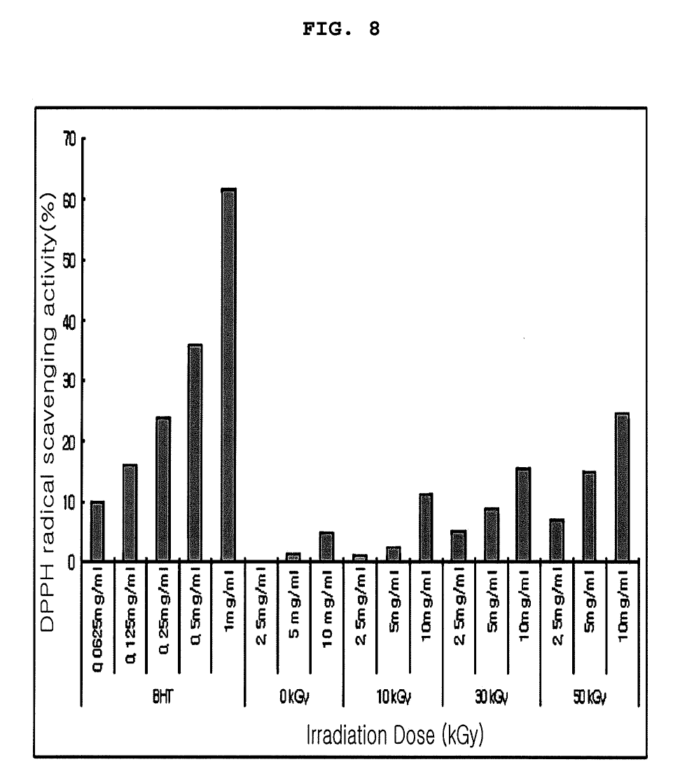

With reference to FIG. 8, the antioxidant activity of gamma ray-irradiated beta-glucan is shown. Over all ranges of radiation dose, gamma ray-irradiated beta-glucan was observed to have a greater DPPH radical-scavenging activity than that of non-irradiated beta-glucan. The experiment results indicate that the antioxidant activity of gamma ray-irradiated beta-glucan increases with an increase in radiation dose.

### Experimental Example 9

### In Vivo Assay of Gamma Ray-Irradiated Beta-Glucan for T Cell Activation

BALB/c mice (6˜7 weeks old) were purchased from Orient Charles River Technology (Seoul, Korea). The mice were housed in polycarbonate cages and maintained at 22±2° C. in environmentally controlled rooms with a 12-h light/dark cycle, with animal diet and water available ad libitum. The animals were divided into 5 groups for purposes of serving as the normal control and for treatment with 0 kGy, 10 kGy, 30 kGy and 50 kGy of gamma radiation. After being irradiated, beta-glucan was orally administered at a dose of 50 mg/kg of body weight to the mice. Immediately after splenectomy, the spleens were maintained in an RPMI medium until splenic analysis. All animal experiments were performed in accordance with the Animal Care Act of the Ministry for Food, Agriculture, Forestry and Fisheries.

Splenic lymphocytes were split from the spleens of female BALB/c mice using a typical method. Splenocytes were maintained at 37° C. in RPMI-1640 medium supplemented with 10% fetal bovine serum, 100 U/mL penicillin and 100 U/mL streptomycin in a 5% CO₂ atmosphere.

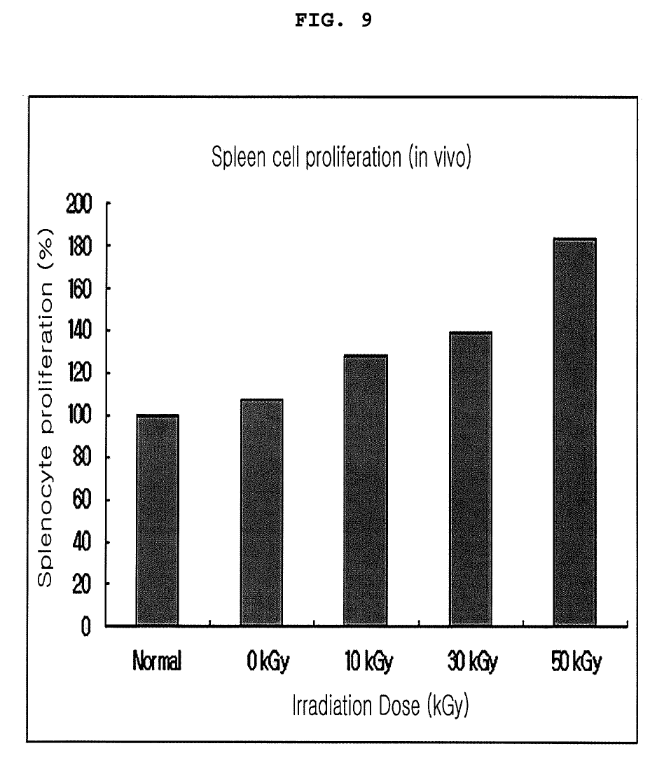

With reference to FIG. 9, spelenocyte activity in mice administered with gamma ray-irradiated or non-irradiated beta-glucan is shown. In order to determine the in vivo immunomodulation activity thereof, splenocytes obtained from mice administered with non-irradiated or irradiated beta-glucan for 7 days were incubated for 24 hours and subjected to MTT analysis. Higher splenic activity was detected in the splenocytes from the mice who had been administered irradiated beta-glucan than those from the mice administered non-irradiated beta-glucan. Splenic activity was measured at 128.5% for mice administered with 10 kGy-irradiated beta-glucan and was significantly increased to 139.3% and 183.8% in the case of mice administered with 30 kGy- and 50 kGy-irradiated beta-glucan, respectively.

### Experimental Example 10

### In Vivo Assay of Gamma Ray-Irradiated Beta-Glucan for Cytokine Secretion

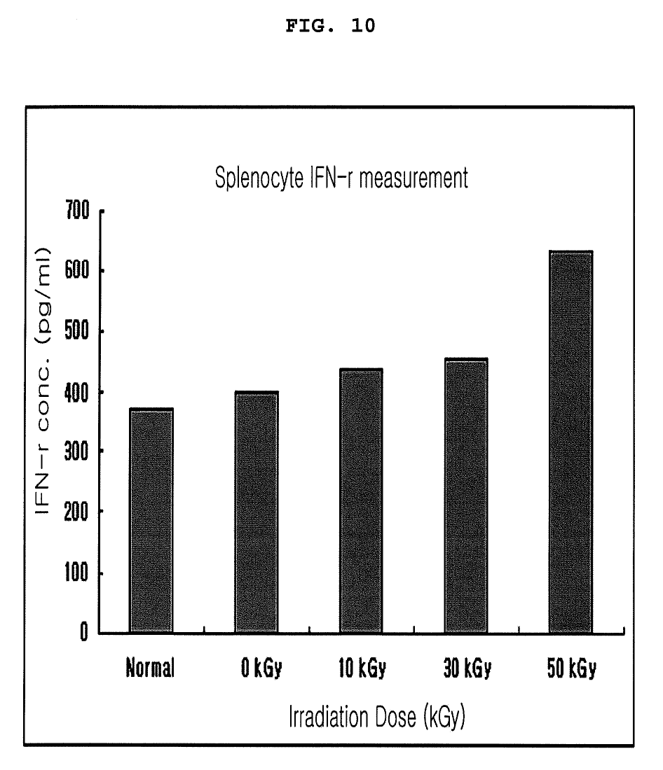

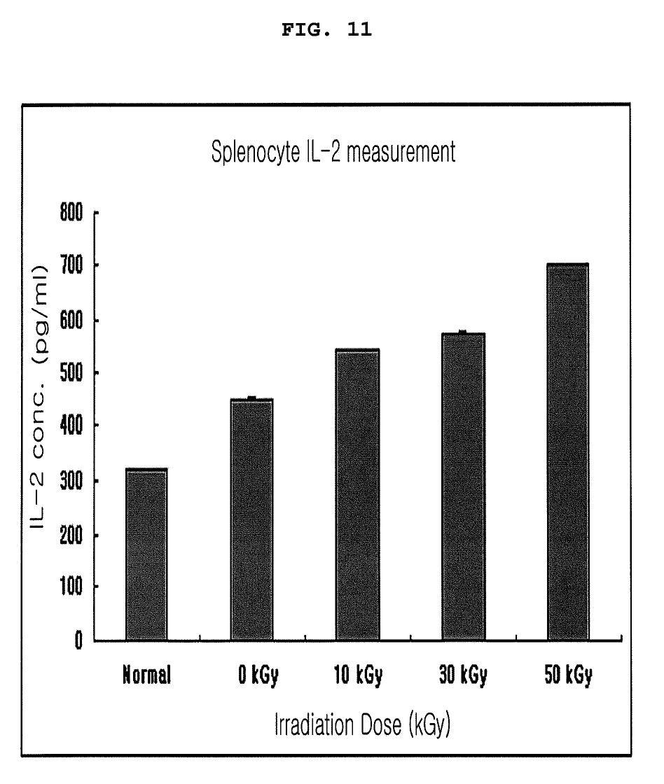

This experiment was conducted to determine the in vivo immunostimulation of non-irradiated and irradiated beta-glucan. For this, Th1-released cytokines were assayed using an ELISA method. In FIGS. 10 and 11, the activity of the splenic Th1 cytokines IFN-γ and IL-2 is shown according to stimulation with normal control and irradiated beta-glucan.

As seen in these figures, the secretion of the Th1 cytokines IFN-γ and IL-2 was significantly increased with an increase of the radiation dose. Larger cytokine activity was detected in the mice administered with 50 kGy-irradiated beta-glucan for 7 days than in the mice administered with 10 or 30 kGy-irradiated beta-glucan.

Formulation examples for low-molecular weight beta-glucan are given herein, below.

### Formulation Example 1

### Preparation of Pharmaceutical Preparation

1. Powder Preparation

| | |
|---|---|
| Low-molecular weight Beta-Glucan: | 2 g |
| Lactose: | 1 g |

The above ingredients were mixed and loaded into an airtight sac to produce a powder agent.

2. Table Preparation

| | |
|---|---|
| Low-molecular weight Beta-Glucan: | 100 mg |
| Corn Starch: | 100 mg |
| Lactose: | 100 mg |
| Mg Stearate: | 2 mg |

These ingredients were mixed and prepared into tablets using a typical tabletting method.

3. Capsule Preparation

| | |
|---|---|
| Low-molecular weight Beta-Glucan: | 100 mg |
| Corn Starch: | 100 mg |
| Lactose: | 100 mg |
| Mg Stearate: | 2 mg |

These ingredients were mixed and loaded into gelatin capsules according to a typical method of producing capsules.

4. Pill Preparation

| | | |
|---|---|---|
| Low-molecular weight Beta-Glucan: | 1 | g |
| Lactose: | 1.5 | g |
| Glycerin: | 1 | g |
| Xylitol: | 0.5 | g |

These ingredients were mixed and prepared into pill forms, each pill weighing 4 g.

5. Granule Preparation

| | |
|---|---|
| Low-molecular weight Beta-Glucan: | 150 mg |
| Soybean Extract: | 50 mg |
| Glucose: | 200 mg |
| Starch: | 600 mg |

These ingredients were mixed in 100 mg of 30% ethanol and dried at 60° C. to give granules which were then loaded into sacs.

### Formulation Example 2

### Preparation of Food

Foods comprising the low-molecular weight beta-glucan of the present invention were prepared as follows.

1. Seasoning Preparation

A seasoning comprising 20˜95 weight parts of the low-molecular weight beta-glucan of the present invention was prepared for use in foods which improve the health.

2. Flour-Based Food Preparation

To 100 weight parts of flour were added 0.5˜5.0 wt parts of the low-molecular weight beta-glucan according to the present invention and the resulting flour mixture was used in breads, cakes, cookies, crackers and noodles.

3. Soup and Gravy Preparation

The low-molecular weight beta-glucan according to the present invention was added in an amount of 0.1˜5 wt parts to 100 weight parts of typical soups or gravies to prepare health-improving soups or gravies for consumption with meat processed products or noodles.

4. Ground Beef Preparation

To 100 wt parts of typical ground beef was added 10 wt parts of the low-molecular weight beta-glucan of the present invention to produce health-improving ground beef.

5. Preparation of Dairy Products

To 100 wt parts of milk was added 5˜10 wt parts of the low-molecular weight beta-glucan according to the present invention and the milk mixture was used in the preparation of various dairy products such as butter and ice cream.

6. Preparation of Zen Food

Unmilled rice, barley, glutinous rice, and unshelled adlay were pre-gelatinized using a typical method, dried, roasted and then ground into a powder with a particle size of 60 meshes.

Black soybean, black sesame and wild sesame were steamed according to a typical method, dried, roasted and then ground into a powder with a particle size of 60 meshes.

The low-molecular weight beta-glucan according to the present invention was concentrated in a vacuum using a vacuum concentrator and dried in a convection oven, followed by grinding into a powder with a particle size of 60 meshes.

The powders made of the grains, the seeds, and the low-molecular weight beta-glucan according to the present invention were formulated at the following ratios to yield a zen food:

Grains (unmilled rice 30 wt parts, unshelled adlay 15 wt parts, barley 20 wt parts),

Seeds (wild sesame 7 wt parts, black soybean 8 wt parts, black sesame 7 wt parts),

Dry powder of the extract according to the present invention (3 wt parts),

Ganoderma lucidum (0.5 wt parts),

Foxglove (0.5 wt parts).

### Formulation Example 3

### Beverage Preparation

1. Healthy Beverage Preparation

| | | |
|---|---|---|
| Low-molecular weight Beta-Glucan: | 1000 | mg |
| Citric acid: | 1000 | mg |
| Oligosaccharides: | 100 | g |
| Japanese Apricot Liquid Extract: | 2 | g |
| Taurine: | 1 | g |

| | |
|---|---|
| Purified water: | added to bring volume to 900 mL |

These ingredients were homogeneously formulated according to a typical method and the formulation was heated at 85° C. for about 1 hour with stirring, loaded into a 2 L bottle, subjected to pasteurization and stored in a refrigerator until use.

This composition is provided as a preferred example suitable for use in beverages, but the contents may be changed depending on regional and national factors, such as consumer classes, countries, etc.

2. Vegetable Juice Preparation

5 g of the low-molecular weight beta-glucan according to the present invention was added to 1,000 mL of typical tomato or carrot juice to yield a health-improving vegetable juice.

3. Fruit Juice Preparation

1 g of the low-molecular weight beta-glucan according to the present invention was added to 1,000 mL of typical apple or grape juice to yield a health-improving fruit juice.

### Formulation Example 4

### Cosmetic Formulation with Low-Molecular Weight Beta-Glucan

The low-molecular weight beta-glucan of the present invention may be used as an active ingredient for an immunity-enhancing, functional cosmetic. In this example, immune enhancement cosmetics were formulated with the low-molecular weight beta-glucan of the present invention to produce emulsion-type cosmetics, such as nutrition lotion, cream, essence, etc., and water-soluble cosmetics, such as skin lotion.

1-1. Preparation of Emulsion-Type Cosmetics

Emulsion-type cosmetics were prepared from the composition given in Table 2 as follows.

1) a mixture of materials 1˜9 was heated at 65˜70° C.

2) material 10 was admixed with the mixture of step (1).

3) a mixture of materials 11˜13 was heated at 65˜70° C. until complete dissolution.

4) while being heated, the mixture of step 3 was slowly added to the admixture of step 2, followed by emulsification at 8,000 rpm for 2˜3 min.

5) material 14 was dissolved in a small amount of water and added to the mixture of step (4), followed by emulsification for 2 min.

6) materials 15˜17 were weighed and added to the mixture of step 5 before additional emulsification for 30 sec.

7) the mixture of step (6) was de-aerated and cooled to 25˜35° C. to produce emulsion-type cosmetics.

TABLE 2
Emulsion-Type Cosmetic Compositions

| | | Emulsion- | Emulsion- | Emulsion- |
|---|---|---|---|---|
| Materials | | Type 1 | Type 2 | Type 3 |
| 1 | Stearic acid | 0.3 | 0.3 | 0.3 |
| 2 | Stearic alcohol | 0.2 | 0.2 | 0.2 |
| 3 | Glyceryl monostearate | 1.2 | 1.2 | 1.2 |
| 4 | Beeswax | 0.4 | 0.4 | 0.4 |
| 5 | Polyoxyethylene sorbitan | 2.2 | 2.2 | 2.2 |
| | monolauric acid ester | | | |
| 6 | Paraoxybenzoic acid | 0.1 | 0.1 | 0.1 |
| | methyl | | | |
| 7 | Paraoxybenzoic acid | 0.05 | 0.05 | 0.05 |
| | propyl | | | |
| 8 | Cetylethylhexanoate | 5 | 5 | 5 |
| 9 | Triglyceride | 2 | 2 | 2 |
| 10 | Cyclomethicone | 3 | 3 | 3 |
| 11 | Distilled water | ~100 | ~100 | ~100 |
| 12 | Conc. Glycerin | 5 | 5 | 5 |
| 13 | Triethanol amine | 0.15 | 0.15 | 0.15 |
| 14 | Polyacrylic acid polymer | 0.12 | 0.12 | 0.12 |
| 15 | Pigment | 0.0001 | 0.0001 | 0.0001 |
| 16 | Perfume | 0.10 | 0.10 | 0.10 |
| 17 | Low-Mw. B-Glucan | 0.0001 | 1 | 10 |

1-2 Preparation of Water-Soluble Type Cosmetics

Water-soluble-type cosmetics were prepared from the composition given in Table 3, as follows.

1) materials 2˜6 were dissolved in material 1 (purified water) using an agitation mixer.

2) materials 8˜11 were completely dissolved in material 7 (alcohol).

3) the solution of step (2) was slowly added to the mixture of step (1) to produce water-soluble type cosmetics

TABLE 3
Water-Soluble Type Cosmetic Compositions

| | | Soluble | Soluble | Soluble |
|---|---|---|---|---|
| Mateirals | | type 1 | type 2 | type 3 |
| 1 | Purified Water | ~100 | ~100 | ~100 |
| 2 | Conc. Glycerin | 3 | 3 | 3 |
| 3 | 1,3-Butylene Glycol | 2 | 2 | 2 |
| 4 | EDTA-2Na | 0.01 | 0.01 | 0.01 |
| 5 | Pigment | 0.0001 | 0.0002 | 0.0002 |
| 6 | Low-Mw. B-Glucan | 0.1 | 5 | 5 |
| 7 | Alcohol (95%) | 8 | 8 | 8 |
| 8 | Para-bezoic acid methyl | 0.1 | 0.1 | 0.1 |
| 9 | Polyoxyethylene | 0.3 | 0.3 | 0.3 |
| | hydrogenated ester | | | |
| 10 | Perfume | 0.15 | 0.15 | 0.15 |
| 11 | Cyclomethicone | — | — | 0.2 |

Exhibiting such physical properties as reduced viscosity and increased water solubility and such biological properties as increased antioxidant activity, splenocyte stimulation and cytokine secretion, as described herein, the low-molecular weight beta-glucans prepared by irradiation in accordance with the present invention find useful application in many fields including the food, medical and cosmetics industries.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A method for preparing low-molecular weight beta-glucan, comprising:
dissolving beta-glucan in a solvent to form a beta-glucan solution;
exposing the beta-glucan solution to an effective amount of radiation; and
recovering a low molecular weight beta-glucan is produced by a degradation of beta-1,3-glucan structure, beta-1,4-glucan structure, and beta-1,6-glucan structure.

2. The method as set forth in claim 1, further comprising drying the solution.

3. The method as set forth in claim 1, wherein the solvent is selected from a group consisting of a buffer, a culture medium, an alcohol, and distilled water.

4. The method as set forth in claim 1, wherein the radiation is selected from a group consisting of a gamma ray, an electron ray, and an X ray.

5. The method as set forth in claim 4, wherein the radiation is a gamma ray.

6. The method as set forth in claim 5, wherein the gamma ray is emitted from a radiation source selected from a group consisting of cobalt (Co)-60, krypton (Kr)-85, strontium (Sr)-90, and cesium (Cs)-137.

7. The method as set forth in claim 6, wherein the gamma ray is emitted from cobalt (Co)-60.

8. The method as set forth in claim 1, wherein the radiation is administered in a dose of 10-100 kGy.

9. The method as set forth in claim 8, wherein the radiation is administered in a dose of 30-50 kGy.

10. The method as set forth in claim 1, wherein the low-molecular weight beta-glucan ranges in molecular weight from 1 to 100 kDa.

11. The method as set forth in claim 10, wherein the low-molecular weight beta-glucan ranges in molecular weight from 25 to 60 kDa.

12. A composition comprising a low-molecular weight beta-glucan, wherein the low molecular weight beta glucan consists of low molecular weight beta-glucan prepared using the method of claim 1.

13. The composition as set forth in claim 12, wherein the low-molecular weight beta-glucan shows a viscosity which is decreased in proportion to an absorbed dose of radiation.

14. The composition as set forth in claim 12, wherein the low-molecular weight beta-glucan shows an increase in water solubility proportional to an absorbed radiation dose.

15. The composition as set forth in claim 12, comprising a low-molecular weight beta-glucan as an active immune enhancing, antioxidant, or atopy therapeutic ingredient.

16. The composition as set forth in claim 15, wherein the low-molecular weight beta-glucan shows T-cell activation activity which is increased proportional to an absorbed dose of radiation.

17. The composition as set forth in claim 16, wherein the low-molecular weight beta-glucan shows spelenic cytokine secretion activity which is increased in proportion to an absorbed dose of radiation.

18. The composition as set forth in claim 17, wherein the cytokine is IFN-γ or IL-2.

19. The composition as set forth in claim 12, wherein said low-molecular weight beta-glucan comprises a distribution of beta-1,3-glucan, beta-1,4-glucan, and beta-1,6-glucan.

20. A method comprising administering to a subject the composition as set forth in claim 12, wherein said administering of the low molecular weight beta glucan composition is effective for enhancing immunity, providing antioxidation treatment, or preventing or treating atopy.

21-23. (canceled)

24. The composition according to claim 12, wherein said composition is an immune-enhancing health food comprising the low-molecular weight beta-glucan as an active ingredient.

25. The method according to claim 20, wherein said administered composition is an immune-enhancing health food comprising said low-molecular weight beta-glucan in combination with other food ingredients.

26. The composition according to claim 12, wherein said composition is a functional cosmetic for preventing and treating atopy, comprising the low-molecular weight beta-glucan as an active ingredient.

27-28. (canceled)

29. The composition of claim 12, wherein said low molecular weight beta glucan ranges in molecular weight from 1 to 100 KDa.

30. The composition of claim 29, wherein said low molecular weight beta glucan ranges in molecular weight from 25 to 60 KDa.

31. The composition of claim 12, wherein said low molecular weight beta glucan is produced by irradiation at a dose of 10-100 kGy.

32. The composition of claim 31, wherein said low molecular weight beta glucan is produced by irradiation at a dose of 30-50 kGy.

33. The composition of claim 12, wherein said low molecular weight beta glucan is produced by irradiation from a radiation source selected from a group consisting of cobalt (Co)-60, krypton (Kr)-85, strontium (Sr)-90, and cesium (Cs)-137.

34. The composition of claim 33, wherein the radiation source is cobalt (Co)-60.

35. The method of claim 20, wherein the low molecular weight beta-glucan comprises a distribution of beta-1,3-glucan, beta-1,4-glucan, and beta-1,6-glucan.

36. The method of claim 20, wherein said low molecular weight beta glucan ranges in molecular weight from 1 to 100 KDa.

37. The composition of claim 36, wherein said low molecular weight beta glucan ranges in molecular weight from 25 to 60 KDa.

38. The composition of claim 20, wherein said low molecular weight beta glucan is produced by irradiation at a dose of 10-100 kGy.

39. The composition of claim 38, wherein said low molecular weight beta glucan is produced by irradiation at a dose of 30-50 kGy.

40. The composition of claim 20, wherein said low molecular weight beta glucan is produced by irradiation from a radiation source selected from a group consisting of cobalt (Co)-60, krypton (Kr)-85, strontium (Sr)-90, and cesium (Cs)-137.

41. The composition of claim 40, wherein the radiation source is cobalt (Co)-60.
